(19) United States
(12) Patent Application Publication
(10) Pub. No.: US 2007/0042054 A1
(43) Pub. Date: Feb. 22, 2007

(54) FUNCTIONAL APHRODISIAC ROLLED HERBAL BIDIS AND CIGARETTES

(75) Inventors: Pushpangadan Palpu, Lucknow (IN); Chandana Venkateshwara Rao, Lucknow (IN); Raghavan Govindarajan, Lucknow (IN); Ajay Kumar Singh Rawat, Lucknow (IN); Sharad Kumar Srivastava, Lucknow (IN); Biswajit Deb, Mumbai (IN); Srinivas Subramanian, Mumbai (IN)

Correspondence Address: OHLANDT, GREELEY, RUGGIERO & PERLE, LLP, ONE LANDMARK SQUARE, 10TH FLOOR, STAMFORD, CT, 06901, US

(73) Assignees: Council of Scientific and Industrial Research; Godfrey Phillips India Limited

(21) Appl. No.: 11/379,449

(22) Filed: Apr. 20, 2006

(30) Foreign Application Priority Data
Apr. 20, 2005 (IN) 0989/DEL/2005

(51) Int. Cl. A61K 36/84; A61K 36/752; A61K 36/54; A61K 36/282; A61K 36/61; A61K 36/53; A61K 36/48; A61K 36/81

(52) U.S. Cl. 424/725; 424/733; 424/757; 424/740; 424/736; 424/739; 424/742; 424/745; 424/751

(57) ABSTRACT

An herbal formulation comprising the synergistic combination of fiber/powder/granules of the aerial parts of at least two plants selected from the group comprising Marticaria chamomilla, Hygrophila auriculata, Aquilaria agallocha, Valeriana wallichii, Nordostachys jatamansi, Artemisia pallen, Papaver somniferum(mainlyleaf), Evolvulus alsinoides, Ficus benghalensis, Echinops echinatus, Lobelia nicotianaefolia, Mucuna pruriens, Tribulus terrestris and along with one or more flavoring agents selected from essential/aromatic oils. Thee composition is aromatic and having pleasant smell, are non-irritants, no itching, and has no toxic-effects, no breathing problems, has no bronchial irritation, no pain in nose, ear and throat, has no headache, not allergic. There are no health hazards or health risk in normal subjects when the person attempts to reduce their use of nicotine or non nicotine bidis and cigarettes or stop using such products altogether. At the same time the composition strengthens the person's immune system allowing that person to define against the respiratory disorders/diseases.

### FIELD OF THE INVENTION

The present invention relates to use of the herbal formulation comprising the synergistic combination of fiber/powder/granules of the aerial parts of at least two plants selected from the group comprising of Marticaria chamomilla, Hygrophila auriculata, Aquilaria agallocha, Valeriana wallichii, Nordostachys jatamansi, Artemisia pallen, Papaver somniferum(mainlyleaf), Evolvulus alsinoides, Ficus benghalensis, Echinops echinatus, Lobelia nicotianaefolia, Mucuna pruriens, Tribulus terrestris along with one or more flavoring agents selected from essential/aromatic oils.

More particularly, it relates to the use of the said formulation in formulated products such as bidis and cigarettes/snuffs/chewing gums.

### DISCUSSION OF THE BACKGROUND ART

For the joy of living, medicated smoke should become a part of the daily routine. When used as therapy, the smoke is inhaled through the nose for head and neck disease, and through the mouth for throat disorders. The potential advantages of delivering a drug to the lung by inhalation have been well known to physicians and drug abusers for years. For drugs that exert their biological effect in the lung, these include rapid onset of action, reduced dose and minimized side effects compared to the same drug delivered by mouth. First line asthma therapy is currently based on inhaled corticosteroids which minimize the problems associated with oral steroids, such as adrenal suppression, electrolyte imbalance and muscle weakness. Tobacco smoke has been used in western culture for more than 400 years, human inhalation of cigarette smoke is a twentieth century phenomenon with major medical and economic consequences. However, excessive smoking is now recognized as one of the major health problems throughout the world. The most important difficulty results from the fact that heavy smokers are dependent on nicotine, which is considered to be one of the risk factors in tobacco smoke. During the combustion of tobacco the important risk factors formed such as carbon monoxide, tar products, aldehydes and hydrocyanic acid (U.S. Pat. No. 5,335,678). US patent 20040103908 describes the herbal cigarette free from the existing tobacco cigarettes contains a predetermined mixture of different herbs packed in the filtered or non-filtered cigarette paper tubes. The U.S. Pat. No. 6,497,234 relates to a mixture of herbs and other natural products that can be used as a healthy substitute for tobacco and also alleviates the addictive effects of nicotine, and provides an aid to quit smoking tobacco-related products. Whereas, the US patent 20050061828 invention relates generally to the field of packaging machines which provides for the packaging of at least two types of smoking products consisting of tobacco cigarettes/cigars and bidis. None of the prior art discloses functional cigarettes having aphrodisiac properties. Accordingly studies were undertaken to develop a functional aphrodisiac rolled herbal bidis, and cigarettes that benefits in innumerable ways the health of our bodies, minds and spirits exist mood lifting or execrating the sexual activity.

The main object of the present invention is to provide the use of the herbal formulation comprising the synergistic combination of fiber/powder/granules of the aerial parts of at least two plants selected from the group comprising of Marticaria chamomilla, Hygrophila auriculata, Aquilaria agallocha, Valeriana wallichii, Nordostachys jatamansi, Artemisia pallen, Papaver somniferum(mainlyleaf), Evolvulus alsinoides, Ficus benghalensis, Echinops echinatus, Lobelia nicotianaefolia, Mucuna pruriens, Tribulus terrestris and along with one or more flavoring agents selected from essential/aromatic oils.

Another object of the present invention is to provide the use of the said herbal formulation in formulated products such as bidis and cigarettes/snuffs/chewing gums.

Further object of the present invention is to provide novel functional aphrodisiac rolled herbal bidis, and cigarettes useful in mood lifting or execrating the sexual activity.

### SUMMARY OF THE INVENTION

The present invention deals with the use of the herbal formulation comprising the synergistic combination of fiber/powder/granules of the aerial parts of at least two plants selected from the group comprising of Marticaria chamomilla, Hygrophila auriculata, Aquilaria agallocha, Valeriana wallichii, Nordostachys jatamansi, Artemisia pallen, Papaver somniferum(mainlyleaf), Evolvulus alsinoides, Ficus benghalensis, Echinops echinatus, Lobelia nicotianaefolia, Mucuna pruriens, Tribulus terrestris along with one or more flavoring agents selected from essential/aromatic oils and in the development of aphrodisiac smoking articles such as cigarettes and bidis. The novelty of the present investigation lies in providing (1) bidi or cigarette exciting sexual desire; (2) treatment of impotency and erectile dysfunction; (3) which helps in the cure or relief from asthma or respiratory diseases and improves the digestion and evacuation of bowel; (4) spreads good aroma to the surrounding and ties up the good relations in business and profession and relief stress.

### DETAILED DESCRIPTION OF THE PREFERRED EMBODIMENT

Accordingly, the present invention provides the use of the herbal formulation comprising the synergistic combination of fiber/powder/granules of the aerial parts of at least two plants selected from the group comprising of Marticaria chamomilla, Hygrophila auriculata, Aquilaria agallocha, Valeriana wallichii, Nordostachys jatamansi, Artemisia pallen, Papaver somniferum(mainlyleaf), Evolvulus alsinoides, Ficus benghalensis, Echinops echinatus, Lobelia nicotianaefolia, Mucuna pruriens, Tribulus terrestris along with one or more flavoring agents selected from essential/aromatic oils.

In an embodiment of the present invention, the said formulation further comprising upto 50% of the said formulation and the rest being the conventional additives along with tobacco.

In another embodiment of the present invention, the synergistic combination of fiber/powder/granules are mixed in the ratio of Marticaria chamomilla (2-15%), Hygrophila auriculata (5-12%), Aquilaria agallocha (1-10%), Valeriana wallichi (2-10%), Nordostachys jatamansi(2-6%), Artemisia pallen (2-15%), Papaver somniferum (0.5-13.5%), Evolvulus alsinoides (2.5-10%), Ficus benghalensis (1-15%), Echinops echinatus (0.5%-10), Lobelia nicotianaefolia (2.8-7.9%), Tribulus terrestris (5-15%) along with one or more specific flavoring agents selected from essential/aromatic oils.

Further, in an embodiment of the present invention, the essential/aromatic oils are selected from the group comprising of clove, eucalyptus, Angelica archangel, Apium graveolens, Cananga odorata, Carum carvi, Citrus aurantifolia, Citrus limonum, Cymbopogon citrates, Cymbopogon martinii, Cinnamomum verum, Lavandula angustifolia, Lavendula officinalis, Melaleuca alternifolia, Myristica fragrans, Pelargonium graveolens, Rosa damascene, Santalum album, Jasminum multiflorum, Jasminum sambac, Ocimum sanctum, Ocimum gratissimum and Nicotiana tabacum.

Yet in an embodiment of the present invention, the use of the said formulation in formulated products such as bidis and cigarettes/snuffs/chewing gums.

Still in an embodiment of the present invention, the said formulated product is effective against erectile dysfunction.

Still in an embodiment of the present invention, the said formulated product induce sexual desire.

Further, in an embodiment of the present invention, the said formulated product helps in removing depression.

Still in an embodiment of the present invention, the said formulated product helps in relieving stress.

Still in an embodiment of the present invention, the said formulated product spreads fragrance or good aroma in surrounding.

Still in an embodiment of the present invention, the said formulated product helps in curing or relieving asthma or breathing difficulties.

Still in an embodiment of the present invention, the said formulated product helps in improving the digestion and evacuation of bowel.

Still in an embodiment of the present invention, the said formulated product scavenges the free radicals and acts as potent antioxidant.

Still in an embodiment of the present invention, the said formulated product is non-irritant to eye, nose and does not possess itching property.

Still in an embodiment of the present invention, the said formulated product is non-irritating, having no toxic-effects, no itching, no breathing problems, no bronchial irritation, and no pain in nose, ear and throat.

Still in an embodiment of the present invention, the said formulated product helps in improving the potent enzymatic superoxide dismutase enhancing activity.

Still in an embodiment of the present invention, the said formulated product helps in defending the body against the effect of stressful environment providing vitality and energy.

The present invention also provides a herbal formulation comprising the synergistic combination of fiber/powder/granules of the aerial parts of at least two plants selected from the group comprising of Marticaria chamomilla, Hygrophila auriculata, Aquilaria agallocha, Valeriana wallichii, Nordostachys jatamansi, Artemisia pallen, Papaver somniferum(mainlyleaf), Evolvulus alsinoides, Ficus benghalensis, Echinops echinatus, Lobelia nicotianaefolia, Mucuna pruriens, Tribulus terrestris along with one or more flavoring agents selected from essential/aromatic oils.

In an embodiment of the present invention, the said formulation further comprising upto 50% of the said formulation and the rest being the conventional additives along with tobacco.

In another embodiment of the present invention, the synergistic combination of fiber/powder/granules are mixed in the ratio of Marticaria chamomilla (2-15%), Hygrophila auriculata (5-12%), Aquilaria agallocha (1-10%), Valeriana wallichi (2-10%), Nordostachys jatamansi(2-6%), Artemisia pallen (2-15%), Papaver somniferum (0.5-13.5%), Evolvulus alsinoides (2.5-10%), Ficus benghalensis (1-15%), Echinops echinatus (0.5%-10), Lobelia nicotianaefolia (2.8-7.9%), Tribulus terrestris (5-15%) along with one or more specific flavoring agents selected from essential/aromatic oils.

Further in an embodiment of the present invention, the essential/aromatic oils are selected from the group comprising of clove, eucalyptus, Angelica archangel, Apium graveolens, Cananga odorata, Carum carvi, Citrus aurantifolia, Citrus limonum, Cymbopogon citrates, Cymbopogon martinii, Cinnamomum verum, Lavandula angustifolia, Lavendula officinalis, Melaleuca alternifolia, Myristica fragrans, Pelargonium graveolens, Rosa damascene, Santalum album, Jasminum multiflorum, Jasminum sambac, Ocimum sanctum, Ocimum gratissimum and Nicotiana tabacum.

Further, the invention provides herbal bidis/cigarettes/snuffs/chewing gums comprising the said herbal formulation.

In an embodiment of the present invention, the said bidis/cigarettes/snuffs/ chewing gums contain low tar content.

The plants used in the preparation of the functional aphrodisiac cigarettes have been reported to have following descriptive characters

Hydrophila auriculata

Botanical Description—A stout herb up to 1.5 m high, more or less hispid with long hairs; stems numerous, erect, usually unbranched, subquadangular, thickened at the nodes. Leaves in whorls of 6, the outer 2 the largest, oblong-lancoelate or oblanceolate, upto 18cm long and 3.2 cm wide, with sharp, yellow, axillary spines; the two outer leaves of each whorl larger and others much smaller. Flowers bluish-purple, in sessile axillary whorls; calyx 4-partite, lobes unequal, clothed with soft hairs; corolla distinctly 2-lipped, the upper2-lobed, the lower 3-lobed. Fruits 0.8 cm long, linear -oblong, pointed, containing 4-8 orbicular seeds. Flowers between October and December in central Indian deciduous forest regions.

Distribution and habitat—Throughout the plains of India, in moist habitats such as marshy margins of canals and rice fields. Also found in tropical Himalayas, Myanmar, Indonesia and Malaysia.

Medicinal properties and uses—In ayurveda, seeds and roots comprise the drug Kolilaksah, a reputed remedy for arthritis. The bitter seeds are considered aphrodisiac and tonic and are used as a uterine sedative for pregnant women, and for treating diseases of biliousness. The seeds are given with sugar or milk in the treatment of impotence, gonorrhoea and spermatorrhoea. A paste of the seeds mixed with buttermilk or whey, is given for diarrhoea. A decoction of the roots is used as a diuretic and to treat rheumatism, gonorrhoea, and other diseases of the genito-urinary tract, jaundice and anasarca. The leaves are considered oleaginous, tonic, aphrodisiac and hypnotic; they are used to treat diarrhea, dysentery, thirst, urinary calculi and urinary discharges, inflammations, constipation, abdominal troubles. Among the rural inhabitant of Bhavnagar district of Gujarat, the whole plant is reportedly used to treat jaundice,dropsy, and rheumatism. In unani system, the plant is known as Talamakhana, the leaves being considered useful for relieving cough; they are applied externally to treat gleet, lumbago and joint pains. In siddha,the flower, seeds and whole plant are used for treating anaemia, dropsy, ascites, constipation, piles and aphrodisiac.

Evolvulus alsinoides

Botanical Description—A prostrate, annual or perennial herb with spreading wiry, silky hairy branches. Leaves alternate, sub-sessile to short-petioled, variable in shape: broadly ovate, elliptic or lanceolate, 0.8-2.6 cm long and up to 1 cm wide, acute to rounded at both ends, hairy on both sides. Flowers blue or white, campanulate to subrotate, 0.5-0.6 cm long and wide, borne 1-3 together on axillary peduncles 0.6-4 cm long, glabrous; seeds usually 4, pale brown to black,ovoid, 1.5-1.7 mm long. Flowers and fruits from July to December.

Distribution and habitat—native to the American tropics, now pantropical in distribution. It is found in open and grassy habitats and thorn scrub in most of the drier parts of India to an elevation of about 1800 m in the Himalayas.

Medicinal properties and uses—The whole plant is considered alternative, anthelmintic, antiphlogistic, bitter, emmenagogue, febrifuge, tonic and vermifuge; it is a well known remedy for bowel complaints, specifically dysentery. In ayurveda the whole plant is used as a brain tonic and to traet nervous debility and dysentery. Mixed with cumin and milk it is used to relieve fever and to treat memory loss, nervous debility,scrofula and syphilis. Among the tribal inhabitants of Maharastra the dried plant powder is taken internally as a brain tonic. Mixed with Ocimum sanctum, it is prescribed for fever associated with diarrhea or indigestion. The plant, mixed with oil, is used to promote hair growth. In the Khedbrahma region of northern Gujarat, the juice of the plant is used as a local application to promote healing of ulcers; it is taken internally by mahalis of bihar to treat hysteria. The leaves are sometimes smoked to relieve asthma and chronic bronchitis. In Madhya Pradesh the leaf paste is applied to the fingers to treat whitlow. The roots are used in ayurvedic practice to treat gastric and duodenal ulcers, and intermittent fever in children; its flowers are reportedly useful for treating uterine bleeding.

Ficus benghalensis

Botanical Description—a very large tree upto 30 m tall,evergreen except in very dry localities, with spreading branches and numerous aerial roots that often develop into accessory trunks helping the tree to spread laterally a considerable distance from the original stem; bark smooth, light grey; twigs light brownish-ochre; branchlets, stipules, petioles, underside of leaves and figs white velvety, slowly turning glabrescent. Leaves spirally arranged on branchlets, upto 10-30 cm long and 7-20 cm wide, coriaceous, elliptic to ovate, apex obtuse, base rounded, with 5-7 basal nerves; petioles 2.5-5 cm long; stipules stout. Fruits sessile, axillary in pairs, 1.3-1.9 cm in diameter, globose, silky-pubescent, scarlet or brick red when ripe. Flowers during the summer and fruits during the rainy season.

Distribution and habitat—Found through out the moist and dry deciduous forest regions and plains of south Asia to an elevation of about 1200 m, from the sub-Himalayan hills in the north through peninsular India and in srilanka. It is widely planted throughout the country and elsewhere in tropical Asia in gardens and along roads for shade.

Medicinal properties and uses—The stem bark, rootbark, aerial roots, leaves, vegetative buds and milky exudates are all used in ayurvedic medicine and in the preparation of number of important compound formulation. The crushed seeds and the milky juice exuded from the cut stems, branches and twigs are applied externally to relieve pains, sores, ulcers, and bruises, and as an anodyne for treating rheumatism and lumbago. It is considered a valuable application for relieving and healing cracked and burning soles, and is also used as a remedy for toothache. The crushed dried fruits are taken with honey as a treatment for spermatorrhoea among the tribal inhabitants of central Orissa; in this region the latex of the plant is taken with banana in the treatment of gonorrhea. The seeds are considered cooling and tonic. A paste of the leaves, or the heated leaves, is applied as a poultice to promote healing of abscesses. An extract of the leaves is used as an aphrodiasiac by men and women in northerneastern Karnataka. The bark is astringent; its infusion is considered a powerful tonic and useful for treating diabetes, dysentery, and diarrhea, leucorrhoea and nervous diseases. An infusion of young buds is considered useful for diarrhea and dysentery. The young tips of the aerial roots are given as an anti-emetic; crushed and boiled in cow's milk, the hot filtered solution is taken to relieve piles among tribal inhabitants of sundergarh district in Orissa. The aerial root tips are also applied as a paste to relieve bleeding piles and to promote healing of syphilitic lesions among the konds of southwestern Orissa and mixed with egg as an external application to promote healing of bonefracture amongs the Gonds of uttarpradesh.

Echinops echinatus

Botanical Description—a rigid, pubescent, annual herb upto 1 tall, with branches widely spreading from the base. Leaves alternate, sessile, 7.5-12.5 cm long, oblong, pinnatifid, covered with cottony wool beneath, the lobes triangular and oblong, sinuate and spiny, the spines often 2.5 cm long. Flower heads white or purple, compact, globose, 2.5-3.8 cm in diameter, clustered at the ends of branches; involucres surrounded by strong white bristles resembling pappus-hairs; pappus short, yellowish, forming a short cylindrical brush above the achene. Achenes 4 mm long, densely villous.

Distribution and habitat—Pantropical in distribution, and found almost throughout India to an elevation of 15 00 m. It is common in fields, grasslands, open forests and roadsides in peninsular India.

Medicinal properties and uses—The plant is considered bitter, tonic and diuretic, and is used to treat hoarse cough, hysteria,dyspepsia, scrofula, and ophthalmia. In ayurveda it si used to treat strangury, biliousness, urinary discharges, gleet and heartdiseases. In unani the plant is considered stomachic, antipyretic and analgesic; it is used to increase appetite stimulate liver function and to treat diseases of the brain, ophthalmia. Chronic fever, joint pain and inflammation. The root are considered aphrodisiac in both unani and ayurvedic medicine. The powdered roots are applied to the hair to destroy lice. Among the Bhils in Rajasthan, the root extract in water is given orally to treat cough and colds. A paste of the roots is applied to relieve scorpion sting among the rural inhabitants of Bhavnagar district in Gujarat. The pounded roots, mixed with water, are taken orally for relief of diarrhea and dysentery by the Kolams of northern Andhra Pradesh. In ayurveda the root is reportedly used to facilitate childbirth.

Tribulus terrestris

Botanical Description—A more or less pubescent annual herb with prostate or decumbent primary branches upto 1.5 cm long radiating from the crown of the taproot; roots slender, cylindrical, somewhat fibrous, 10-15 cm long, light brown and faintly aromatic. Leaves opposite, paripinnate, upto 5.5 cm long, one of each pair usually smaller that the other; leaflets 3-6 pairs, 6-12 mm long, oblong to linear-oblong, with silky hairs on both surfaces, apex mucronate, base rounded oblique; petiolules very short. Flowers pale-yellow to yellow, 0.7-2 cm in diameter, leaf-opposed, solitary ; pedicles 1.2-2 cm long, slender, hairy; sepals 6 mm long, lanceolate, acute; petals 1 cm long, oblongobovate, claw short, hairy. Fruits globose, consisting of 5-12 woody cocci, each with 2 pairs of hard, sharp, divaricate spines, one pair longer than the other; seeds several in each coccus with transverse partitions between them. Flowers and fruits throughout the year.

Distribution and habitat—Native to the Mediterranean region; introduced and naturalized nearly throughout the tropics, subtropics and warm temperate regions of the world. It is common weed found throughout India, particularly in drier regions on sandy soils, to an altitude of approximately 5400 m in Kashmir.

Medicinal properties and uses—The fruits are credited with diuretics, cooling, demulcent, tonic and aphrodisiac properties. They are used in ayurveda to treat kidney stones, painful urination and other genitor-urinary disorders, usually in the form of an infusion; they are also prescribed in the treatment of diadetes, piles, rheumatism, dropsy, breathing diffulcuties,heart disease, impotence, and bright's disease. The leaves are considered to possess stomachic properties, and a paste prepared from them is used to treat bladder stones. The root is considered to have aperient and tonic properties, and is a constituent of the ayurvedic drugs dasamula and amrithaprasa ghritha; the fruits are used in a number of important compound ayurvedic preparations.

Mucuna pruriens

Botanical Description: Pantropical in distribution, it is found almost throughout India and in the Andaman and Nicobar Islands in damp habitats, ravines and scrub forests. Occasionally cultivated. A large, pubescent, herbaceous or sometimes woody, annual climber. Leaves trifoliate, leaf rachis 5-12 cm long, appressed silky; stipules linear, about 4 mm long; leaflets membranous, 7.5-12.5 cm long and 5-7.5 cm wide, ovate-rhomboid, asymmetrical, apex obtuse or acute,base rounded or truncate,both surfaces grey silky-pubescent. Flower purple, borne in pendant, axillary, 6-30 flowered racemes 15-30 cm long, the flowers solitary or 2-3 together along a slender silky rachis; pedicels 3-6 mm long,hairy; bracteoles 8 mm long, hairy; calyx 1 cm long, silky with a few irritant bristles,tube carnpanulate,upper teeth connate into a triangular lip as long as the tube, lateral teeth lanceolate and as long as the tube; corolla 2.5-3.7 cm long, keel slightly incurved. Fruit (pod) S-shaped, thick, turgid, 5-10 cm long and 1.2-1.8 cm wide, longitudinally ribbed, densely covered with yellow-brown stinging hairs; seeds 4-6,black, ovoid,1.2 cm long. Flowers and fruits almost throughout the year but mainly between August and January in central Indian deciduous forest regions.

Medicinal properties and uses: The plant is highly valued in traditional Indian medicine. The seeds and the hairs covering the pods are considered a powerful aphrodisiac in Ayurveda medicine; they are also used to treat impotence, leucorrhoea and urinary troubles. The roots are tonic; stimulant and diuretic.seeds are used as an anthelmintic, nerve tonic and also in scorpion sting.

Phytochemistry: M. pruriens seeds have been reported to be a good source of 3,4-dihydroxyphenylalanine (L-dopa). Damodaran and Ramaswamy (1937) have reported the isolation of L-dopa from seeds in a yield of 1.5 percent. The alcoholic extract of M. pruriens seeds gave four alkaloids viz., mucunine, mucunadine, Prurienine and prurieninine The 80 percent alcoholic extract of seeds showed the presence of 5 indolic compounds, two of which were identified as tryptamine and 5-hydroxytryptamine. Different parts of M. pruriens except trichomes of pods yielded four indole-3-alkylamines. Choline was reported in all parts of the plant. The trichomes of pods gave only 5-hydroxytryptamine, whereas the stem-leaf showed the presence of 6-methoxyharman. M. pruriens seed oil was found to contain stearic, palmitic, myristic, arachidic, oleic, linoleic acid and a sterol.

Pharmacology: The claim of Ayurvedic physician that M. pruriens is effective in the treatment of parkinsonism, has prompted many pharmacologists and clinicians to investigate its effect on central nervous system. The powdered seed extract of M. pruriens was devoid of anti cholinergic activity. The extract showed hypotensive action in dogs and spasmodic action in guinea pig preparation, the effect being blocked by mepyramine maleate and not by atropine sulphate. The extract had no effect on frog rectus, but revealed a histaminergic activity. The observation suggested that the seed powder may act by some mechanism other than through anti cholinergic property in parkinsonism. Various fractions obtained from M. pruriens seeds were evaluated against oxotremorine-induced tremors in mice and reserpine-induced rigidity, hypokinesia and catatonia in rats. The result indicated that the extract had no anti cholinergic activity but had a potent anti-parkinsoniam effect which was not however, entirely due to L-dopa. The L-dopa free fraction of the seed showed significant anti parkinsonian activity at a dose of 200 mg/kg.

Papaver somniferum

Part used: Leaves

Botanical Description

Cultivated for production of opium which is harvested by incising the capsules at a particular stage known as “industrial maturity”; the product of first lancing contains higher percentage of morphine. Opium used as a narcotic, sedative, anodyne, antispasmodic, hypnotic, and sudorific. Whole opium is much less used, its pure alkaloids, morphine and codeine and their salts are preferred.

Medicinal Properties and Uses

Eating of opium creates a sense of euphoria and is habit forming; consumed orally, by smoking, and by injection. Poppy seed oil used for culinary purposes, free from narcotic action; also used in dairrhoea and dysentery and in compositions for skin care. Capsules contain the same constituents as opium; infusion applied as a soothing application. Extract used as a sedative against irritating cough.

Aquilaria agallocha

Botanical Description

Source of agar, which consists of irregular patches of dark wood highly charged with oleoresin, found in the interior of comparatively old trees. It yields an essential oil, called chuwah or agar attar, used in perfumery.

Medicinal Properties and Uses

Agar is considered as a stimulant, cordial, tonic and carminative. It is now used mainly for making fumigators and pastilles. Wood used for cabinets and inlay work. Bark yields a fibre used for ropes; it was used in Assam for home made paper.

Valeriana wallichi

Botanical Description

Rhizomes and roots of this species are known as Indian valerian as distinct from those of valerian officivnalis, called valerian. Both the drugs are used for the same purposes.

Medicinal Properties and Uses

They are used as incense and in perfumes; also used medicinally for hysteria, hypochondriasis, nervous unrest, and emotional troubles, and as a carminative. Pulverized drug is mixed with sugar and prescribed in urinary troubles. Decoction used as a sedative after parturition. Rhizomes and roots yield an essential flavour for tobacco, honey, etc; also used as a tonic and stimulant. Drug has yielded a new group of iridoid or monoterpenic derivatives known as a valepotriates, used as transquillizers and sedatives in formulation similar to meprobromate.

Artemisia pallen

Source of danvana oil, with cisdavanone as a main constituent; used in high grade perfumes.

Medicinal Properties and Uses

Plant accredited with anthelmintic, tonic, and antipyretic properties and also considered a good fodder.

The composition of essential oil is extracted from leaves, resins and fibre. The composition is formulated as products mainly in the form of bidis and cigarettes. The composition is aromatic and having pleasant smell, are non-irritants, no itching, and has no toxic-effects, no breathing problems, has no bronchial irritation, no pain in nose, ear and throat, has no headache, not allergic. There are no health hazards or health risk in normal subjects when the person attempts to reduce their use of nicotine or non nicotine bidis and cigarettes or stop using such products altogether. At the same time the composition strengthens the person's immune system allowing that person to define against the respiratory disorders/diseases.

The following examples are given by way of illustration of the present invention and should not be construed to limit the scope of present invention.

### EXAMPLE 1

HERBAL FORMULATIONS

| | |
|---|---|
| Plant name | Percentage composition |

F1

| | |
|---|---|
| Nicotiana tabacum | 50 |
| Matricaria chamomilla | 15 |
| Aquilaria agalocha | 10 |
| Valeriana wallichii | 10 |
| Artemisia pallens | 15 |
| Flavouring agent | Quantity Sufficient |

F2

| | |
|---|---|
| Nicotiana tabacum | 50 |
| Artemisia pallens | 15 |
| Papaver somniferum | 5 |
| Hygrophila auricular | 10 |
| Evolvulus alsinoidesta | 10 |
| Ficus benghalensis | 10 |
| Flavouring agent | Quantity Sufficient |

F3

| | |
|---|---|
| Nicotiana tabacum | 50 |
| Artemisia pallens | 15 |
| Echinops schinatus | 10 |
| Lobelia nicotianaefolia | 5 |
| Mucuna pruriens | 5 |
| Tribulus terrestris | 15 |
| Flavouring agent | Quantity Sufficient |

### EXAMPLE 2

Blood Pressure Recording

Blood pressure (Systolic and diastolic) and pulse rate were recorded after 1^{st} and 2^{nd} week of smoke exposure using a tail cuff blood pressure recorder (UGO Basile, Model no 58500; Comerio-varese, Italy). Rats were acclimatized to heating chamber (29-30° C.) for 30 min before recording the blood pressure (between 8. 30 and 10.30 AM), in order to cause a sufficient vasodilatation in the caudal artery. Three recordings were measured for each rat and the average was calculated.

TABLE 1
Body weight, systolic, diastolic pressure
and pulse rate of smoke exposure in rats

| | Control rats | 1^{st} week | 2 nd week |
|---|---|---|---|
| Body weight (g) | 325 ± 2.8 | 316 ± 3.4 | 307 ± 3.3 |
| Systolic blood pressure | 116.8 ± 1.24 | 119.5 ± 1.20 | 117.8 ± 1.22 |
| (mm Hg) | | | |
| Diastolic blood pressure | 77.87 ± 1.05 | 78.77 ± 1.26 | 83.30 ± 1.31 |
| (mm Hg) | | | |
| Pulse rate (beats/min) | 387.0 ± 25.3 | 397.90 ± 24.8 | 411.2 ± 26.5 |

The result explains that there a change in the vital parameters on the blood pressure and pulse rate. However, there is a tendency in reduction of the body weight. This may be due to the reduction in the intake of the food during the course of exposure of the smoke.

Mounting Behaviour

To quantify mounting behaviour, non-oestrous female mice were paired with males treated with single or repeated doses of the smoke. Animals were observed for 4 h and their behaviors were scored blindly as described (Nelson et al 1995). Briefly males were placed individually in a clear aquarium. Then a non-oestrous female was introduced into the arena. Animals remained paired for 4 h. The numbers of mounts were recorded, during a 15 min observation period for smoke exposure at the start of each hour. All experiments were performed from 09.00 to 13.00 h on sunny days (room temperature, 25-27° C.). A mount was operationally defined as the male assuming the copulatory position but failing to achieve intromission. Intromission was defined as the male's penis entering the vagina in association with thrusting behavior.

TABLE 2
Effect of a smoke for 7 and 14 days
on sexual behaviour of male mice

| | 7 days exposure | | 14 days exposure | |
|---|---|---|---|---|
| | Number of mounts/15 min | | Number of mounts/15 min | |
| Group | 1 h | 2 h | 1 h | 2 h |
| Control | 4.2 ± 1.2 | 1.5 ± 0.6 | 4.5 ± 1.3 | 1.2 ± 0.2 |
| Smoke | 5.6 ± 1.3 | 1.8 ± 0.6 | 5.5 ± 1.4 | 1.6 ± 0.3 |
| exposure | | | | |
| (Twice daily) | | | | |

Values are mean ± SEM, number of animals in each group is 6.

The results states that there is tendency in increase in the number of mounts at 1 h of exposure of the smoke compared with that of the control.

The present formulation is effective against erectile dysfunction and to induce sexual desire. It is also useful to remove stress, spreads fragrance or good aroma in surrounding. It helps in improving the digestion and evacuation of bowel and also provides vitality and energy.

What is claimed is:

1. Use of the herbal formulation comprising fiber/powder/granules of the aerial parts of at least two plants selected from the group comprising of: Marticaria chamomilla, Hygrophila auriculata, Aquilaria agallocha, Valeriana wallichii, Nordostachys jatamansi, Artemisia pallen, Papaver somniferum(mainlyleaf), Evolvulus alsinoides, Ficus benghalensis, Echinops echinatus, Lobelia nicotianaefolia, Mucuna pruriens, Tribulus terrestris, and one or more flavoring agents selected from essential or aromatic oils.

2. Use of the herbal formulation as claimed in claim 1 further comprising up to 50% of said herbal formulation and the rest being the conventional additives along with tobacco.

3. Use of the herbal formulation as claimed in claim 1, wherein said fiber/powder/granules are mixed in the ratio of Marticaria chamomilla (2-15%), Hygrophila auriculata (5-12%), Aquilaria agallocha (1-10%), Valeriana wallichi (2-10%), Nordostachys jatamansi(2-6%), Artemisia pallen (2-15%), Papaver somniferum (0.5-13.5%), Evolvulus alsinoides (2.5-10%), Ficus benghalensis (1-15%), Echinops echinatus (0.5%-10), Lobelia nicotianaefolia (2.8-7.9%), Tribulus terrestris (5-15%) along with one or more specific flavoring agents selected from essential or aromatic oils.

4. Use of the herbal formulation as claimed in claim 1, wherein the essential or aromatic oils are at least one selected from the group comprising of: clove, eucalyptus, Angelica archangel, Apium graveolens, Cananga odorata, Carum carve, Citrus aurantifolia, Citrus limonum, Cymbopogon citrates, Cymbopogon martini Cinnamomum verum, Lavandula angustifolia, Lavendula officinalis, Melaleuca alternifolia, Myristica fragrans, Pelargonium graveolens, Rosa damascene, Santalum album, Jasminum multiflorum, Jasminum sambac, Ocimum sanctum, Ocimum gratissimum and Nicotiana tabacum.

5. Use of the herbal formulation as claimed in claim 1 in formulated products such as bidis and cigarettes/snuffs/chewing gums.

6. Use of the herbal formulation as claimed in claim 5, wherein the said formulated product is effective against erectile dysfunction.

7. Use of the herbal formulation as claimed in claim 5, wherein the said formulated product induce sexual desire.

8. Use of the herbal formulation as claimed in claim 5, wherein the said formulated product helps in removing depression.

9. Use of the herbal formulation as claimed in claim 5, wherein the said formulated product helps in relieving stress.

10. Use of the herbal formulation as claimed in claim 5, wherein the said formulated product spreads fragrance or good aroma in surrounding.

11. Use of the herbal formulation as claimed in claim 5, wherein the said formulated product helps in curing or relieving asthma or breathing difficulties.

12. Use of the herbal formulation as claimed in claim 5, wherein the said formulated product helps in improving the digestion and evacuation of bowel.

13. Use of the herbal formulation as claimed in 5, wherein the said formulated product scavenges the free radicals and acts as potent antioxidant.

14. Use of the herbal formulation as claimed in claim 5, wherein the said formulated product is non-irritant to eye, nose and does not possess itching property.

15. Use of the herbal formulation as claimed in claim 5, wherein the said formulated product is non-irritating, having no toxic-effects, no itching, no breathing problems, no bronchial irritation, and no pain in nose, ear and throat.

16. Use of the herbal formulation as claimed in claim 5, wherein the said formulated product helps in improving the potent enzymatic superoxide dismutase enhancing activity.

17. Use of the herbal formulation as claimed in claim 5, wherein the said formulated product helps in defending the body against the effect of stressful environment providing vitality and energy.

18. A herbal formulation comprising fiber/powder/granules of the aerial parts of at least two plants selected from the group consisting of: Marticaria chamomilla, Hygrophila auriculata, Aquilaria agallocha, Valeriana wallichii, Nordostachys jatamansi, Artemisia pallen, Papaver somniferum(mainlyleaf), Evolvulus alsinoides, Ficus benghalensis, Echinops echinatus, Lobelia nicotianaefolia, Mucuna pruriens, Tribulus terrestris, and one or more flavoring agents selected from essential or aromatic oils.

19. A herbal formulation as claimed in claim 18, further comprising up to 50% of said herbal formulation and the rest being the conventional additives along with tobacco.

20. A herbal formulation as claimed in claim 18, wherein said fiber/powder/granules are mixed in the ratio of Marticaria chamomilla (2-15%), Hygrophila auriculata (5-12%), Aquilaria agallocha (1-10%), Valeriana wallichi (2-10%), Nordostachys jatamansi(2-6%), Artemisia pallen (2-15%), Papaver somniferum (0.5-13.5%), Evolvulus alsinoides (2.5-10%), Ficus benghalensis (1-15%), Echinops echinatus (0.5%-10), Lobelia nicotianaefolia (2.8-7.9%), Tribulus terrestris (5-15%) along with one or more specific flavoring agents selected from essential or aromatic oils.

21. A herbal formulation as claimed in claim 18, wherein the essential or aromatic oils are at least one selected from the group comprising of: clove, eucalyptus, Angelica archangel, Apium graveolens, Cananga odorata, Carum carve, Citrus aurantifolia, Citrus limonum, Cymbopogon citrates, Cymbopogon martinii, Cinnamomum verum, Lavandula angustifolia, Lavendula officinalis, Melaleuca alternifolia, Myristica fragrans, Pelargonium graveolens, Rosa damascene, Santalum album, Jasminum multiflorum, Jasminum sambac, Ocimum sanctum, Ocimum gratissimum and Nicotiana tabacum.

22. Herbal bidis/cigarettes/snuffs/chewing gums comprising said herbal formulation as claimed in claim 18.

23. Herbal bidis/cigarettes/snuffs/chewing gums as claimed in claim 22, wherein the said bidis/cigarettes/snuffs/chewing gums contain low tar content.
